(19) United States
(12) Patent Application Publication
(10) Pub. No.: US 2013/0225953 A1
(43) Pub. Date: Aug. 29, 2013

(54) CONCURRENT STIMULATION EFFECT DETECTION

(71) Applicants: Antonio Oliviero, Toledo (ES); Guglielmo Foffani, Madrid (ES); Ivan Panyavin, Toledo (ES); Laura Mordillo-Mateos, Plasencia (ES); Juan De Los Reyes Aguilar Lepe, Toledo (ES); Anna Merzagora, Philadelphia, PA (US); Kambiz Pourrezaei, Galdwynne, PA (US); Mauricio Rodriguez, Philadelphia, PA (US)

(72) Inventors: Antonio Oliviero, Toledo (ES); Guglielmo Foffani, Madrid (ES); Ivan Panyavin, Toledo (ES); Laura Mordillo-Mateos, Plasencia (ES); Juan De Los Reyes Aguilar Lepe, Toledo (ES); Anna Merzagora, Philadelphia, PA (US); Kambiz Pourrezaei, Galdwynne, PA (US); Mauricio Rodriguez, Philadelphia, PA (US)

(73) Assignees: FUNDACION HOSPITAL NACIONAL DE PARAPLEJICOS, TOLEDO (ES); DREXEL UNIVERSITY, PHILADELPHIA, PA (US)

(21) Appl. No.: 13/581,122

(22) PCT Filed: Feb. 25, 2011
(86) PCT No.: PCT/US11/26268

§ 371 Date: Dec. 5, 2012

Related U.S. Application Data
(60) Provisional application No. 61/308,426, filed on Feb. 26, 2010.

(51) Int. Cl. A61B 5/00

(52) U.S. Cl. CPC A61B 5/4848
USPC 600/323; 607/45; 600/9

(57) ABSTRACT

Functional near infrared spectroscopy (fNIRS) imaging is utilized to measure the influence of stimulation in real time. An fNIRS imaging device is integrated into a transcranial direct current stimulation (tDCS) stimulator by embedding fNIRS optodes on the tDCS stimulating electrodes. During tDCS stimulation, blood oxygenation levels are measured. For example, concentrations of oxyhemoglobin and deoxyhemoglobin are measured and compared to the concentrations of a baseline resting state to provide feedback on the efficacy of tDCS. Based on the feedback, a tDCS threshold and a dose-response relation for a particular subject can be quantified, and individualized stimulation parameters can be determined for the particular subject.

### CROSS-REFERENCE TO RELATED APPLICATIONS

This application claims the benefit of U.S. Patent Application Ser. No. 61/308,426, filed Feb. 26, 2010, which is incorporated by reference herein in its entirety.

### TECHNICAL FIELD

The technical field generally relates to detecting the effect of stimulation and more specifically relates to concurrently stimulating and detecting the stimulation effect in real time.

### BACKGROUND

Transcranial direct current stimulation (tDCS) is a noninvasive brain stimulation technique for the treatment of many neurological or neuropsychiatric disorders. These disorders include neuropathic pain, depression, Alzheimer's disease, stroke-induced aphasia, and stroke-induced motor and sensory dysfunction. The main effect of tDCS is to modulate the cortical excitability. An indicator of excitability change in the cortex is variation in regional blood flow and energy metabolism. However, investigations on regional blood flow and energy metabolism of the brain have been limited, as brain within the skull is not easily accessible for performing experimental procedures.

Existing systems for tDCS cannot effectively measure the direct cortical effects of tDCS. Techniques for studying the metabolic and/or functional state of the brain, such as photon emission-computed tomography (PET), functional magnetic resonance (fMRI), and electroencephalography (EEG), are only capable of measuring the after-effects of tDCS. In addition, these existing techniques are invasive, extremely expensive, not portable, or lack the ability to detect changes in cortical excitability and spatial localization.

Due to these deficiencies, currently, the application of tDCS uses the standard parameters on all subjects. For example, the same intensity is applied to each subject, despite that the scalp and skull have different effect on the tDCS efficacy on different subjects, in different head locations and at different ages. These drawbacks have significantly affected the clinical efficacy of tDCS and restrained tDCS from entering the clinical practice as treatment for a variety of conditions. Accordingly, there is a need for a portable, non-invasive and cost-effective way to quantify the subject-to-subject response variability to stimulation in real time.

### SUMMARY

Currently while stimulating, an influence of stimulation is detected in a noninvasive manner using functional near-infrared spectroscopy (fNIRS). fNIRS is utilized to measure the oxygenation state of hemoglobin of a subject. According to one embodiment, while stimulating the central nervous system of a subject, near-infrared light reflected from the subject is detected. An influence of the stimulating is then determined based on the detected reflected near-infrared light. For example, the detected near-infrared light is indicative of regional blood oxygenation level in the subject, and the influence of the stimulating can be measured by comparing the regional blood oxygenation level with a baseline regional blood oxygenation level.

According to one embodiment, stimulation is administered via a stimulating portion, such as a stimulating electrode configured to deliver transcranial direct current stimulation (tDCS). Functional brain monitoring is achieved by using one or more functional near-infrared (fNIRS) light sources and one or more light detectors which are fixed through the stimulating electrode. The light detector may be adapted for detecting reflected near-infrared light indicative of concentrations of oxyhemoglobin (oxy-Hb) and deoxyhemoglobin (deoxy-Hb) in regional tissues. Accordingly, in one embodiment, an fNIRS light source and an fNIRS detector are embedded in a tDCS stimulating electrode for monitoring a response to tDCS stimulation at the same time as delivering tDCS stimulation.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not limited to implementations that solve any or all disadvantages noted in any part of this disclosure.

### BRIEF DESCRIPTION OF THE DRAWINGS

The following detailed description of example embodiments is better understood when read in conjunction with the appended drawings. For the purposes of illustration, there is shown in the drawings exemplary embodiments; however, the subject matter is not limited to the specific elements and instrumentalities disclosed. In the drawings:

FIG. 1 is a diagram of non-limiting, exemplary system for concurrently stimulating and detecting.

FIG. 2 illustrates example placements of a device for concurrently stimulating and detecting.

FIG. 3 illustrates an example embodiment of a device for concurrently stimulating and detecting.

FIG. 4 depicts an example embodiment of fNIR spectroscopy.

FIGS. 5A and 5B illustrate example embodiments of a device for concurrently stimulating and detecting.

FIG. 6 illustrates another example embodiment of a device for concurrently stimulating and detecting.

FIG. 7 illustrates another example embodiment of a device for concurrently stimulating and detecting.

FIG. 8 illustrates another example embodiment of a device for concurrently stimulating and detecting.

FIG. 9 is a flow diagram of non-limiting, exemplary process for concurrently stimulating and detecting.

FIG. 10 is a block diagram of an example system for concurrently stimulating and detecting.

### DETAILED DESCRIPTION OF ILLUSTRATIVE EMBODIMENTS

Disclosed herein are techniques for concurrently stimulating and detecting an influence of stimulation in a quantitative and noninvasive manner. In one embodiment, transcranial direct current stimulation (tDCS) is used to stimulate a subject. tDCS is a noninvasive stimulation technique for the treatment of many neurological or neuropsychiatric disorders. The main effect of tDCS is to modulate the cortical excitability. An indicator of excitability change in the cortex is variation in regional blood flow (rCBF) and energy metabolism. According to an example embodiment, functional near-infrared spectroscopy (fNIRS) is used to evaluate stimulation effect by monitoring variations in rCBF and metabolism.

fNIRS offers a noninvasive, repeatable, and portable measure of regional blood oxygenation state in brain cortical tissue. An active brain consumes oxygen transported to the brain by oxy-Hb in the blood. As the oxy-Hb gives up oxygen, the oxy-Hb transforms into deoxy-Hb. Therefore, changes in deoxy-Hb and oxy-Hb concentration provide an index of blood oxygenation and hence rCBF and metabolic variations. As deoxy-Hb and oxy-Hb have different absorption spectra in the visible and NIR wavelength ranges, spectroscopy techniques such as fNIRS can be used to measure changes in deoxy-Hb and oxy-Hb concentration. For example, fNIRS can measure the hemodynamic changes related to the human brain activities, such as motor, visual, auditory, language, and other cognitive functions. Additionally, fNIRS can detect even small changes in the cerebral hemodynamic changes. Accordingly, fNIRS can be used to measure concentrations of deoxy-Hb and/or oxy-Hb to quantitatively evaluate the effect of stimulation without invasive intervention.

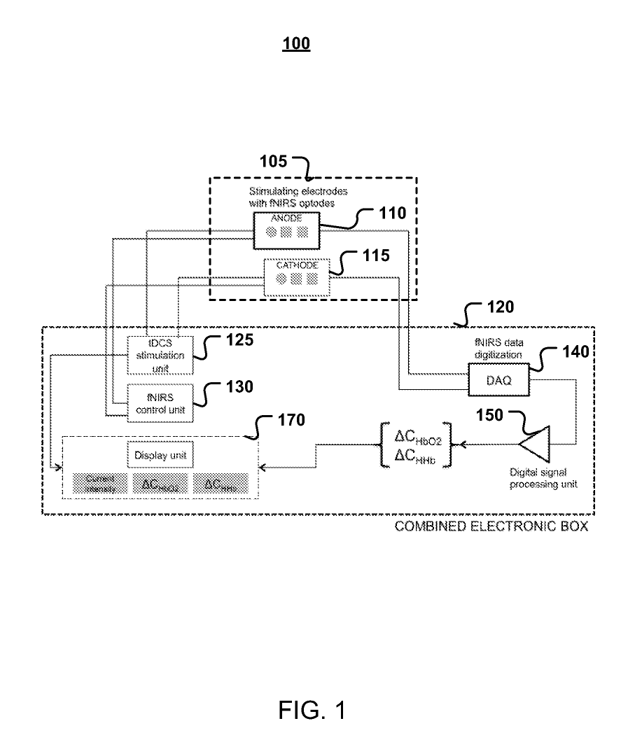

FIG. 1 illustrates an example embodiment of a system for concurrently stimulating and detecting. As shown in FIG. 1, an exemplary system 100 includes a device 105, a stimulation control unit 125, an fNIRS control unit 130, a data acquisition unit 140, a processing unit 150, and/or a display unit 170.

According to an example embodiment, the device 105 is capable of stimulating a subject via tDCS. For example, the device 105 includes one or more tDCS electrodes such as electrode 110 and electrode 115. For example, the electrode 110 is an anode and the electrode 115 is a cathode. The electrodes 110 and 115 can be operatively coupled to the stimulation control unit 125, the light control unit 130, and/or the data acquisition unit 140 by a connection such as wires, cables, fiber optical lines, and/or wireless connection.

In alternative embodiments, the device 105 can include one or more stimulating devices such as intracranial stimulation, transcranial magnetic stimulation (TMS), repetitive transcranial magnetic stimulation (rTMS), deep brain stimulation (DBS), cranial electrotherapy stimulation (CES), electroconvulsive therapy (ECT), functional electrical stimulation (FES), magnetic seizure therapy (MST), vagus nerve stimulation (VNS), transcutaneous electric nerve stimulation (TENS), or other neuromodulation techniques and mechanisms.

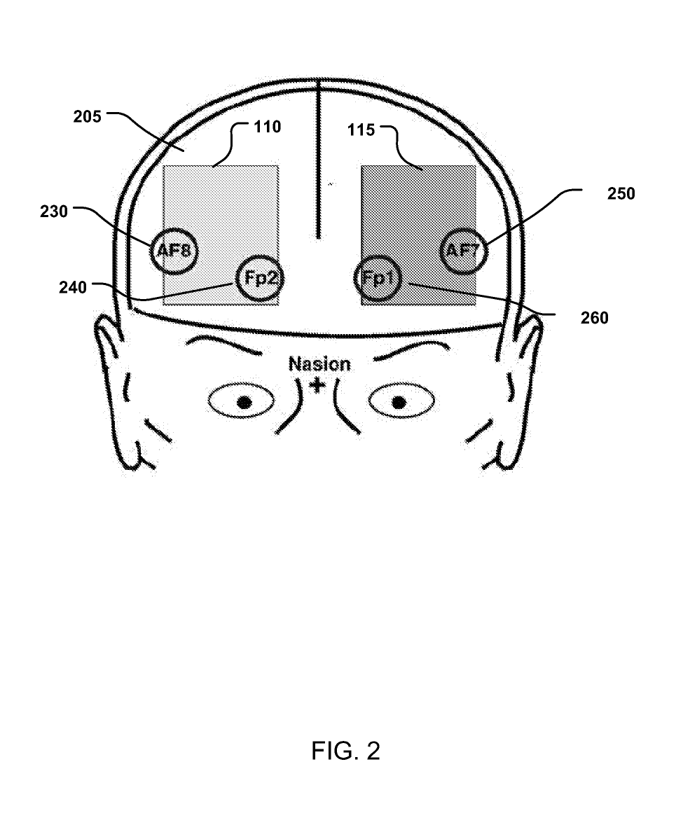

FIG. 2 illustrates example placements of the device 105. In an example embodiment, system 100 can be used to stimulate a subject, such as subject 205. As shown in FIG. 2, the electrodes 110 and 115 are placed on the scalp of subject 205 to deliver stimulation, such as tDCS. In one embodiment, the electrodes 110 and 115 are applied bilaterally at two prefrontal locations. As shown, anode electrode 110 is lateral to Fp1 260 and/or AF7 250, and cathode electrode 115 is lateral to Fp2 240 and/or AF8 230 of the 10-20 international system for EEG electrodes placement, for example. According to one embodiment, the electrodes 110 and 115 include a specially-designed tape for fastening to the forehead. The electrodes 110 and 115 can be further held securely in place with an elastic strap.

According to one embodiment, the anode electrode 110 delivers anodal stimulation, and the cathode electrode 115 delivers cathodal stimulation with a constant current. For example, when applying tDCS to a subject such as the subject 205, anodal polarization increases excitability measures of cortex underlying the electrode, whereas cathodal stimulation reduces excitability.

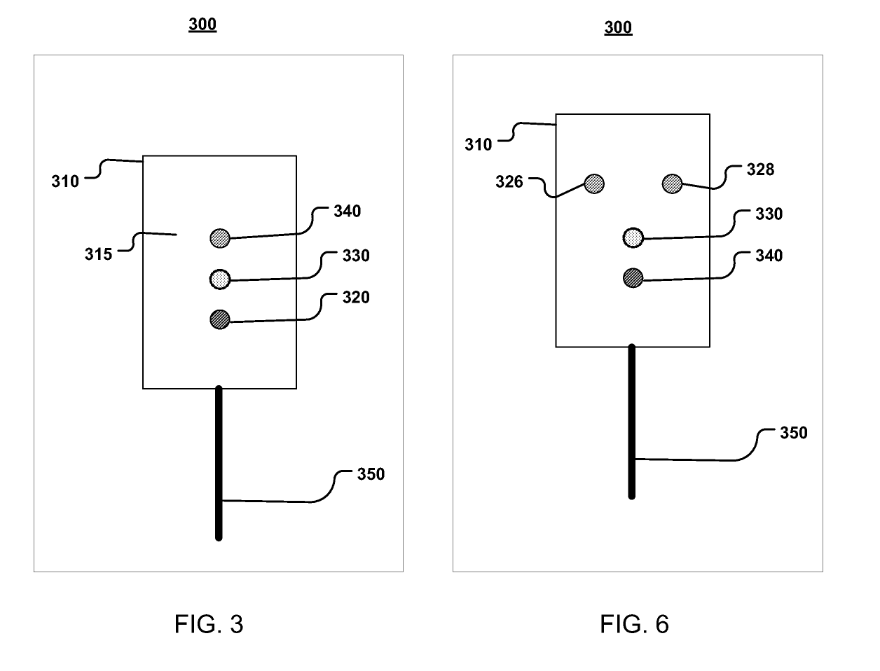

FIG. 3 illustrates an example embodiment of a device for concurrently stimulating and detecting. As shown in FIG. 3, the device 300 includes three portions, a first portion, a second portion, and a third portion. For example, the first portion includes one or more electrodes such as electrode 310 that may produce direct current stimulation. The second portion includes one or more light sources such as light source 320 that provides near-infrared light. The third portion includes one or more light source-detectors such as skin-artifact detector 330 and light detector 340 that may receive reflected near-infrared light. In addition, the device 300 may include a cable such as cable 350. In one embodiment, the various portions are integrated by operable connecting to one another. In an embodiment, the device 300 may use direct current, alternative current, and/or other signals that may stimulate a tissue of a subject.

In another embodiment, the device 300 includes two portions. One portion produces stimulation such as the electrode 310, and another portion measures the subsequent hemodynamic changes in the underlying neural substrate. For example, the electrode 310 is a tDCS electrode. According to one aspect of the embodiment, the measuring portion further includes a transmitting portion that provides near-infrared light such as the light source 320, and a detecting portion that receives reflected near-infrared light such as light detectors 330 and 340. In one embodiment, the various portions are integrated by operable connecting to one another.

According to one embodiment, the electrode 310 provides stimulation with a constant current, for example, of 1 mA. For example, the electrode 310 can deliver current in a range between 0.5 mA to 4 mA, inclusive. According to one aspect of the embodiment, the electrode 310 delivers stimulation in a fade in and fade out fashion, with an interval of, 0.1 s, 2-s, 4-s, 8-s, 10-s, 30-s, 60-s, or the like. According to one aspect of the embodiment, the electrode 310 delivers stimulation in an oscillatory fashion, with a frequency 0.001-Hz, 0.03-Hz, 0.5-Hz, 1-Hz, 13-Hz, 100-Hz, 1005-Hz, or the like. Thus, stimulation can be administered on a subject through the electrode 310.

In one embodiment, the light source 320 is configured to emits light. The light source 320 can be a light emitting diode (LED), a laser diode, or the like that produces light emissions. While FIG. 3 shows the light source 320 is located on the electrode 310, it is to be understood that the light source 320 can be placed remotely.

According to one embodiment, the light source 320 emits NIR light that may mostly be attenuated by oxy-Hb and/or deoxy-Hb. The light source 320 is capable of emitting light of multiple wavelengths. For example, the light source 320 emits light in the NIR spectrum between 700-900 nm. In this range, water, a major component of most tissues, absorbs very little energy, and the spectra of oxy-Hb and deoxy-Hb are distinct enough to allow spectroscopy and measures of separate concentrations of both oxy-Hb and deoxy-Hb molecules. In one embodiment, light source 320 emits a relatively constant intensity during a stimulation period. In another embodiment, the light source 320 emits two or more wavelengths of the NIR light in a range between 700 nm and 900 nm. For example, the light source 320 emits a first wavelength of 730 nm and a second wavelength of 850 nm. The two or more wavelengths of the NIR light are attenuated by deoxy-Hb or oxy-Hb.

As shown in FIG. 3, the device 300 includes one or more light detectors such as light detector 340. The light detector 340 can be a photodetector, a sensor, or the like that detects near-infrared light. In an example embodiment, the light detector 340 is configured to receive light having a wavelength within the band of 700 to 900 nm, inclusive.

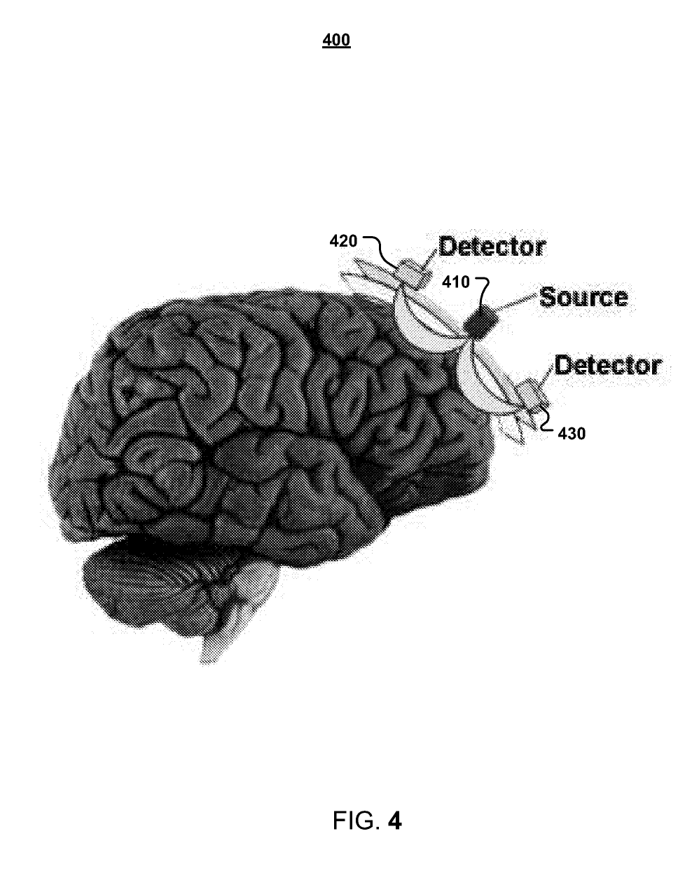

For example, the light detector 340 receives an fNIR signal irradiated by the light source 320 after the fNIR signal has interacted with a tissue of the subject 205, which is described below with reference to FIG. 4.

FIG. 4 depicts an example embodiment of fNIR spectroscopy. fNIR spectroscopy employs specified wavelengths in the optical window that easily pass through most tissue, but reflect back from oxy-Hb and deoxy-Hb. Because photons scatter in a relatively predictable pattern, they can be measured using light detectors such as detectors 420 and 430 on or near the surface of the skin. The relative levels of absorption and backscatter from oxy-Hb and deoxy-Hb provide information about neural activity in the cortex.

In one example embodiment, light source 410 emits NIR light at a frontal cortex of the subject 205. For example, the emitted NIR light are directed at or near the scalp of the subject 205. According to one embodiment, the NIR light emitted by the light source 410 passes through layers of tissue and is absorbed and scattered by oxy-Hb and deoxy-Hb. As shown in FIG. 4, a predictable quantity of NIR light follows a “banana-shaped” path and leaves the tissue. The light detectors, such as detectors 420 and 430, can measure the NIR light after it emerges out of issue.

Turning back to FIG. 3, in one embodiment, the light detector 340 is configured to receive an fNIR signal emitted by the light source 320 after the fNIR signal has passed through a tissue of the subject 205. As described above with respect to FIG. 4, the light detector 340 can measure the light intensity of the NIR light that are not absorbed by cortex tissue. The light detector 340 can also to detect a relative change in the percentage of deoxy-Hb and/or oxy-Hb in a total blood volume.

According to one embodiment, the light source-detector separation provides appropriate NIR light penetration to the brain cortical tissue. For example, the light detector 340 is placed at a distance of 2-4 cm from the light source 320, or at another distance such that the outer layers of the cerebral cortex can be monitored. This distance may enable the light detector 340 to receive NIR light that travels along a banana shaped path between the light source 320 and the light detector 340. In an example embodiment, the depth of imagery is equal to approximately half the distance between the light source 320 and the light detector 340. In this embodiment, because light attenuation increases exponentially with increasing depth of penetration, the maximal depth for imagery for cognitive, emotional, or motivational activity is about 2-3 cm, which allows imagery of much of the cortical surface. Accordingly, brain functions including motor and visual activation, auditory stimulation and the performance of various cognitive, emotional, and/or motivational tasks can be assessed using fNIRS.

In one embodiment, an additional source-detector pair with closer separation may be added for the removal of skin-related artifacts. For example, the device 300 can include another light detector such as the skin-artifact detector 330. The skin-artifact detector 330 may be a photodetector, a sensor, or the like. For example, the light detector 340 is configured to receive NIR light that may contain non-cortical signals and/or artifacts. The received NIR light can contain various wavelengths of fNIR signals emitted by, for example, as the light source 320. The light detector 340 may receive NIR light that may contain a non-cortical signal indicative of an artifact. Artifacts within the received NIR light may have been caused by strong surgery room lighting, patient-table tilting, patient intubation and extubation, or the like. These artifacts may affect fNIRS imaging. According to one embodiment, the skin-artifact detector 330 is placed at a distance of 0.5-1.5 cm from the light source 320, or the like such that a non-cortical signal indicative of an artifact can be captured. According to one aspect of the embodiment, the non-cortical signal captured at the skin-artifact detector 330 is used to discount artifacts or the like that may contaminate the signal acquired by the light detector 340.

In another embodiment, the device 300 also includes a reference signal detector for capturing a reference signal. This reference signal can be used to remove noise from the fNIR signals to measure oxy-Hb and/or deoxy-Hb. For example, the reference signal is emitted to capture non-cortical signals during a period of minimal activation. The non-cortical signal can be used to calibrate and/or remove noise from the other received fNIR signals light. In one example embodiment, the reference signal detector may be placed at a distance from the light source 320 that differs from the distance between light detector 340 and the light source 320.

As shown in FIG. 3, the device 300 includes a cable such as cable 350 that operatively connects electrode 310 to a device such as the electronic box 120 that includes the stimulation control unit 125, the light control unit 130 and the data acquisition unit 140 shown in FIG. 1. In one embodiment, the electrode 310 receives power supply from the electronic box 120 via the cable 350. For example, power for the electrode 310 is provided by one or more batteries through the cable 350. For example, power for the electrode 310 is provided by an alternating current source. For example, power for the light detector 340 and/or the light source 320 may be provided by the electronic box 120. In other embodiments, the cable 350 connects the electrode 310 to the stimulation control unit 125 such that the intensity, polarity, and/or pulse frequency of the electrode 310 can be controlled by the stimulation control unit 125. In another embodiment, the cable 350 connects the electrode 310 to a light control unit such as the light control unit 130. For example, the light control unit 130 can control the wavelength of the light emitted at the light source 320. In another embodiment, the cable 350 connects the electrode 310 to the data acquisition unit 140. The device 300 can provide and/or receive signals via wireless and/or wired connections.

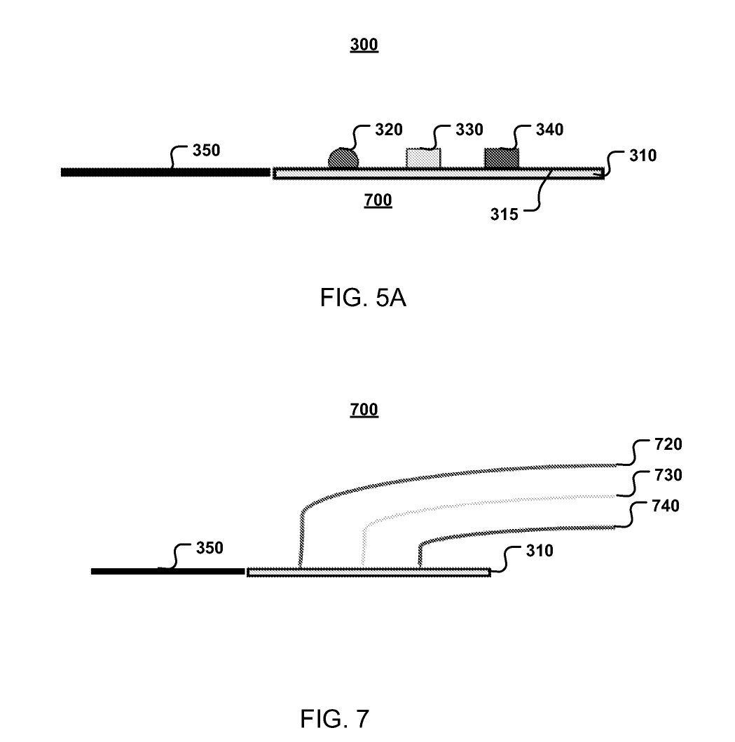

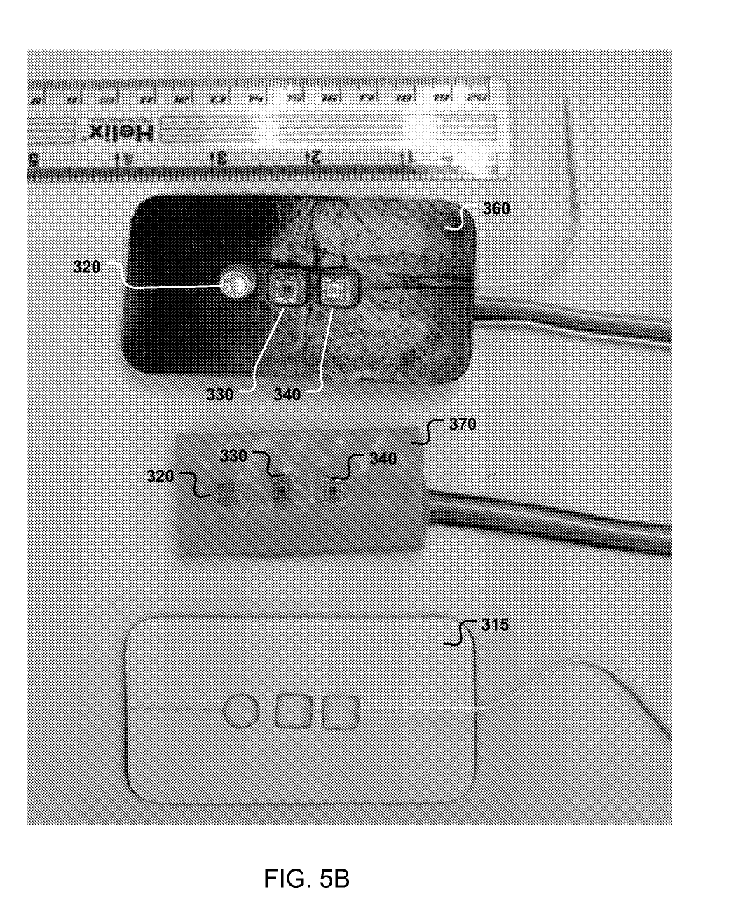

FIG. 5A illustrates the example embodiment of the device 300 from a different perspective. The electrode 310 includes a flexible pad 315 that conducts electrical current. The flexible pad 315 can be made of various materials, for example, highly conductive silicon, saline-soaked synthetic surface sponge, conductive rubber or the like such that the electrode can adapt to the various contours of a subject such as the subject 205. For example, the flexible pad 315 allows the electrode 310 to be placed in such a way that the light detectors are able to maintain close contact with the skin surface, be optically coupled to the skin surface, or the like. This orientation may dramatically improve light coupling efficiency and signal strength. The flexible pad 315 may vary in size, depending on a given application and needed area of coverage. For example, the flexible pad 315 may be approximately 1 cm×1 cm, 1 cm×2 cm, 5 cm×10 cm, 5 cm×5 cm, 10 cm×10 cm or the like. In various embodiments, the flexible pad 315 can be shaped in square, rectangle, triangle, round, or another shape. The flexible pad 315 can have the thickness of, for example, 0.1 cm, 0.3 cm, 0.5 cm, 0.7 cm, 1 cm or the like.

FIG. 5B illustrates example embodiment of the device for concurrently stimulating and detecting. As shown, the device includes a light source 320 and light detectors 330 and 340 that are affixed onto a flexible circuit board. The device includes a flexible pad 315 that conducts electrical current. The flexible pad 315 may be shaped such that when the flexible circuit board is placed on top of the flexible pad 315, the light source 320 and the light detectors 330 and 340 may be exposed. For example, the flexible pad 315 may include three holes such that the light detectors 330 and 340 may directly contact the subject when the device is placed on the subject.

In an embodiment, the light source 320 and light detectors 330 and 340 are hermetically sealed. As shown in FIG. 5B, the device may include a insulating cast 370 that may enclose the flexible circuit board. As shown, in an embodiment, the insulating cast 370 may enclose the flexible circuit board and leave the light source 320 and the light detectors 330 and 340 exposed. For example, the insulating cast 370 may be made of silicon. For example, the light source 320 and light detectors 330 and 340 may be sealed such that cross-talk between the light source 320 and light detectors 330 and 340 and the flexible pad 315 that conducts electrical current may be reduced.

As shown in FIG. 5B, the device may include a conductive gel layer 360. For example, the conductive gel layer 360 may cover the flexible pad 315 such that the device 300 may adhere to the subject. The conductive gel layer 360 may be a thin layer with high viscosity such that spilling over of the gel layer 360 to the light source 320 and light detectors 330 and 340 may be reduced or prevented. The conductive gel layer 360 may be affixed to the flexible pad 315 such that the gel layer 360 may not obstruct the light path between the light source 320 and light detectors 330 and 340. For example, as shown, in an embodiment, the conductive gel layer 360 may leave the light source 320 and the light detectors 330 and 340 exposed such that the light source 320 and the light detectors 330 and 340 may directly contact the subject when the device is placed on the subject. In an embodiment, the conductive gel layer 360 may be a built-in layer of the flexible pad 315.

FIG. 6 illustrates another example embodiment of a device for concurrently stimulating and detecting. As shown in FIG. 6, the device 300 includes more than one light detector, such as light detector 326 and light detector 328. In this embodiment, concentrations of oxy-Hb and/or deoxy-Hb in multiple areas can be measured simultaneously, thereby detecting influence of the stimulating on multiple brain functions.

FIG. 7 illustrates another example embodiment of a device 700 for concurrently stimulating and detecting. As shown, the device 700 includes one or more electrodes such as the electrode 310, one or more light source guides such as light source guide 720, one or more optical guides such as light detection guide 740 and skin-artifact detection guide 730, and/or a cable such as cable 350. The optical guides 720, 730, and 740 can be fiber optic light guides, liquid light guides, combination of/with optical lenses or the like. In one embodiment, the light source guide 720 provides near-infrared light, the light detection guide 740 receives reflected near-infrared light, and the skin-artifact detection guide 730 receives near-infrared light that may contain non-cortical signals and/or artifacts.

As shown in FIG. 7, one end of the light source guide 720 is optically connected to the electrode 310. The other end of the light source guide 720 is operationally connected to a remotely located light source such as a laser diode, a light emitting diode (LED), or the like that is configured to provide light, such as near-infrared light. According to one aspect of the embodiment, a light source is placed remotely, for example, in the electronic box 120 shown in FIG. 1. Hybrid embodiments are also possible, with either light sources or light detectors being placed remotely and optically connected to the electrode.

According to one embodiment, the light detection guide 740 and the skin-artifact detection guide 730 includes a light responsive transducer such as a photodiode that is operative to sense light intensity. As shown in FIG. 7, one end of the light detection guide 740 is optically connected to the electrode 310. The other end of the light detection guide 740 is operatively connected to a light transducer, in turn connected to the data acquisition unit 140 shown in FIG. 1. Similarly, one end of the skin-artifact detection guide 730 is optically connected to the electrode 310. The other end of skin-artifact detection guide 730 is operatively connected to the data acquisition unit 140 shown in FIG. 1.

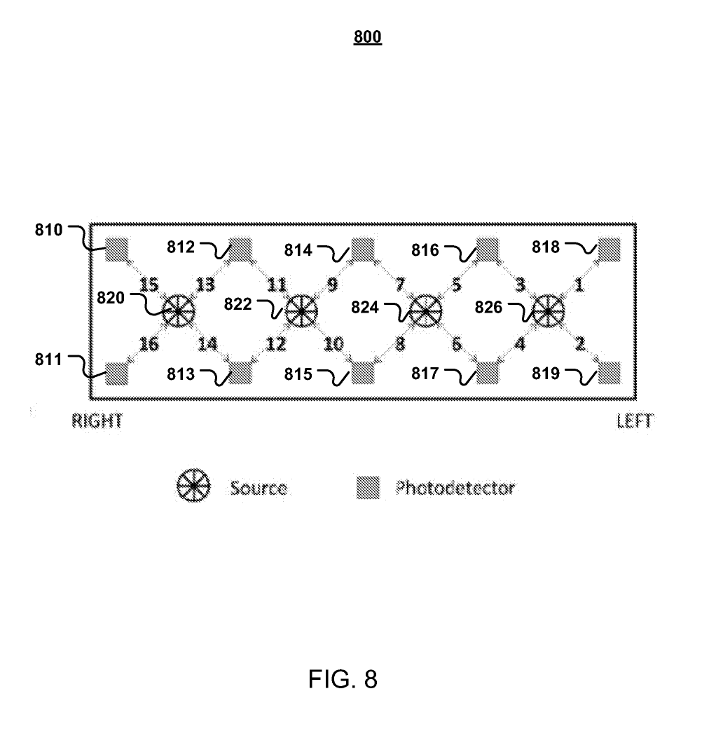

FIG. 8 illustrates another example embodiment of a device 800 for concurrently stimulating and detecting. As shown in FIG. 8, the device 800 includes an electrode such as electrode 310 and multiple light sources and multiple light detectors. In one embodiment, the device 800 includes two rows of light detectors 810-819 and one row of light sources 820-826 positioned between the two rows of light detectors. Each of the light detectors 810-819 can comprise a photodetector, a light sensor, or the like such that light at one or more near-infrared wavelengths can be received. Each of the light sources 820-826 is configured to provide light at a specific wavelength in the near-infrared range. According to one embodiment, the four light sources 820-826 are each activated in turn: each source shines light with input intensity land the four light detectors surrounding the currently active source measured the intensity of the emerging light.

According to one aspect of an embodiment, the light source-detector separation provides appropriate near-infrared (NIR) light penetration to the brain cortical tissue. For example, the source-detector separation can be approximately 2.5 cm, providing a penetration depth of approximately 1.25 cm. An additional source-detector pair with closer separation may be added for the removal of skin-related artifacts. In one embodiment, the device 800 is placed on the scalp of the subject 205 such that the bottom row is closer to the orbitofrontal area and the top row covers more caudal regions of the prefrontal cortex. Each light source-light detector pair (labeled 1 through 16 respectively) forms an optode. Accordingly, this exemplary arrangement of light sources and light detectors yields a total of 16 active optodes and can image cortical areas that correspond to, for example, the dorsal and inferior frontal cortices. Any appropriate configured can be utilized, such as utilizing more or less detectors per optode, more or less sources per optode, and/or utilizing more or less optodes per device, for example.

According to one embodiment, to reduce the dimensionality of the data, the signals from the pairs of light detectors that shared the same degree of laterality but belonged to different rows can be averaged. This operation leads to a total of eight channels. Going from the one at the far left to the one to the far right, the channels are obtained by averaging optodes 1-2, 3-4, 5-6, 7-8, 9-10, 11-12, 13-14, and 15-16 respectively.

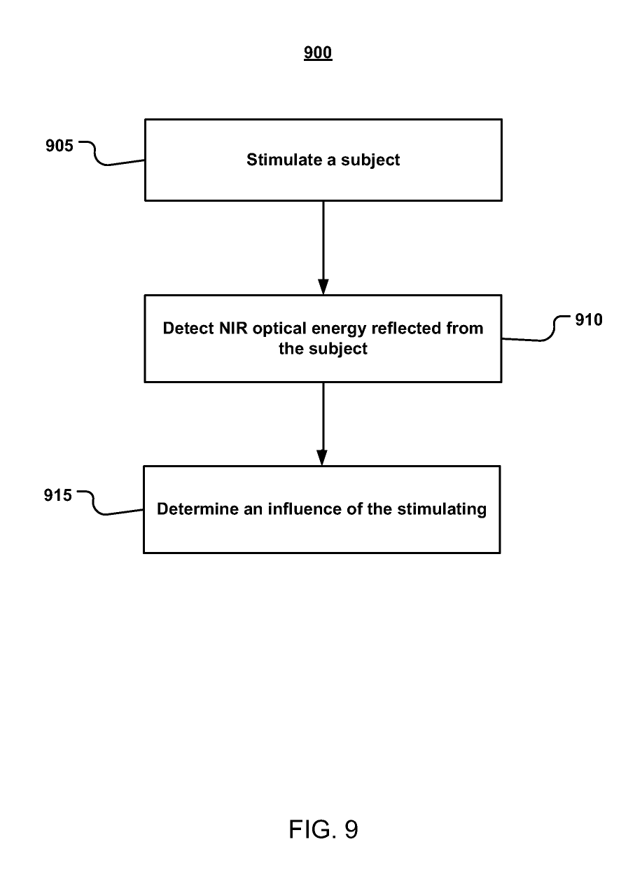

FIG. 9 is a flow diagram of non-limiting, exemplary process 900 for concurrently stimulating and detecting. At 905, a subject is stimulated. According to various embodiments, the subject may be a human being, a brain of a human being, an animal, a brain of an animal, and/or other part(s) of a human being or an animal. For example, the central nervous system of a subject such as the subject 205 can be stimulated by tDCS using the device 105 shown in FIG. 1.

At 910, currently while stimulating, near-infrared light reflected from the subject is detected. In one embodiment, near-infrared light is fNIRS showing regional blood oxygenation level of, for example, a cortical tissue of the subject 205. As described above, the device 105 can be positioned proximate to the scalp of the subject 205. The device 105 directs near-infrared light toward the scalp of the subject. For example, a light source such as the light source 320 emits near-infrared light at or near the scalp of the subject 205. A light detector such as the light detector 340 detects the reflected near-infrared light after it passes through a cortical tissue of the subject.

At 915, an influence of the stimulating on the subject is determined based on the detected reflected near-infrared light. As described above, changes in the rCBF and metabolic variations can indicate the influence of stimulation. And changes in the rCBF and metabolic variations can be detected by measuring concentrations of deoxy-Hb and/or oxy-Hb. In one embodiment, fNIRS is used to measure concentrations of deoxy-Hb and/or oxy-Hb to assess the regional blood oxygenation level of a subject such as the subject 205. In other embodiments, other values indicative of rCBF can be measured.

According to one embodiment, deviations of concentration values of deoxy-Hb and/or oxy-Hb from baseline values are measured. For example, baseline values can be measured in a calming environment before stimulation, or after a predetermined period of time after stimulation. In another example, the baseline values for the subject 205 can be stored in a computer-readable memory and be retrieved at a later time. The baseline values provide an understanding of the deoxy-Hb and/or oxy-Hb levels on a subject-by-subject basis before stimulation is administered. The baseline values can represent the deoxy-Hb concentration levels, the oxy-Hb concentration levels, or a ratio such as a ratio of deoxy-Hb to oxy-Hb or the like that are indicative of blood oxygenation level.

As shown in FIG. 1, the electronic box 120 includes a data acquisition unit such as the data acquisition unit 140. According to one embodiment, the data acquisition unit 140 converts or transduces near-infrared light received at a light detector, such as the light detector 340 on the electrode 110 or the electrode 115, to electrical energy. According to another embodiment, the data acquisition unit 140 converts near-infrared light into digital values for processing, for example, at a processing unit such as processing unit 150. In an embodiment, the electronic box 120 is capable of transmitting data via a wired or wireless connection to a computing device.

In one embodiment, changes in light absorption measured by fNIRS are converted to changes in the concentration of deoxy-Hb and/or the concentration of oxy-Hb. Concentrations of oxy-Hb and/or deoxy-Hb can be determined by measuring an optical density change of NIR light. As described above, NIR light may be directed on a patient, and an fNIR signal may be received. The received fNIR signal may be used to measure an optical density change of the NIR light. Using the optical density change, a deoxy-Hb concentration and/or an oxy-Hb concentration and/or a total hemoglobin volume may be obtained. For example, the processing unit 150 receives the digital values indicative of the light attenuation from the data acquisition unit 140, and computes the changes in concentration of deoxy-Hb and/or the concentration of oxy-Hb compared to the baseline values, which will be described in more detail below.

According to one embodiment, the attenuation that the NIR light undergoes when traveling through a tissue reflects a linear superimposition of two processes, absorption and scattering, which can be represented as:

$${OD}_{λ} = {log}_{10} \frac{{I}_{0 , λ}}{{I}_{λ}} = {A}_{λ} + {S}_{λ} . \qquad ( 1 )$$

where OD_{λ} represents the light attenuation at the wavelength λ expressed in optical density (OD) units, I_{0,λ} is the intensity of the input light at the wavelength λ, I_{λ} is the intensity of the light at the wavelength λ measured by a light detector such as the light detector 340, and A_{λ} and S_{λ} represent the light attenuation caused by absorption and scattering at the wavelength λ respectively.

In the near-infrared region, deoxy-Hb and oxy-Hb are the two main chromophores. Thus, when taking into account the contribution of deoxy-Hb and oxy-Hb to light absorption, the term A_{λ} in Eq. (1) can be written as

A_{λ}=(ε_{HbO2,λ}·C_{HbO2}+ε_{HHb.λ}·C_{HHb})·r_{sd}·DPF₈₀ (2)

based on the modified Beer-Lambert law (mBLL). In Eq. (2), HbO2 represents oxy-Hb, and HHb represents deoxy-Hb, ε_{HbO2} and ε_{HHb} are the specific absorption coefficients of oxy-Hb and deoxy-Hb respectively at the wavelength λ, C_{HbO2} and C_{HHb} represent the concentrations of oxy-Hb and deoxy-Hb respectively in the sampled volume of tissue, r_{sd} is the physical source-detector separation, and DPF_{λ} represents the differential path length factor at the wavelength λ. DPF_{λ} corrects the r_{sd} to give a better estimate of the real length of the path traveled by photons as a consequence of scattering. The values for ε_{HbO2}, ε_{HHb}, r_{sd} and DPF_{λ} are considered time-independent and spatially constant for forehead of an adult subject.

Turning back to Eq. (1), S_{λ} is generally considered a constant factor, dependent on the geometry. Thus, differential representation of mBLL, or changes in light absorption can be written as:

$$\begin{matrix}Δ {OD}_{λ} ( t ) = {OD}_{λ} ( t ) - {OD}_{λ , control} \\ = {log}_{10} \frac{{I}_{λ , control}}{{I}_{λ} ( t )} = \\ = ( {A}_{λ} ( t ) + {S}_{λ} ) - ( {A}_{λ , control} + {S}_{λ} ) \\ = {A}_{λ} ( t ) - {A}_{λ , control} = \\ = ( {ɛ}_{HbO 2 , λ} \cdot Δ {C}_{HbO 2} ( t ) + {ɛ}_{HHb , λ} \cdot Δ {C}_{HHb} ( t ) ) \cdot {r}_{sd} \cdot {DPF}_{λ}\end{matrix} \qquad ( 3 )$$

with ΔC_{HbO2}(t) and ΔC_{HHb}(t) representing the differences in the oxy-Hb and deoxy-Hb concentrations between the instant in time t and the baseline values.

In one embodiment, the differential mBLL is used, and light attenuation is measured at two or more wavelengths in continuous-wave fNIRS in order to calculate ΔC_{HbO2}(t) and ΔC_{HHb}(t). For example, light attenuation is measured at two wavelengths such as 730 nm and 850 nm and ΔC_{HbO2}(t) and ΔC_{HHb}(t) are computed through the solution of two equations:

$$\{ \begin{matrix}Δ {OD}_{730 n m} ( t ) = ( {ɛ}_{HbO 2 , 730 n m} \cdot Δ {C}_{HbO 2} ( t ) + {ɛ}_{HHb , 730 n m} \cdot Δ {C}_{HHb} ( t ) ) \cdot {r}_{sd} \cdot {DPF}_{730 n m} \\ Δ {OD}_{850 n m} ( t ) = ( {ɛ}_{HbO 2 , 850 n m} \cdot Δ {C}_{HbO 2} ( t ) + {ɛ}_{HHb , 850 n m} \cdot Δ {C}_{HHb} ( t ) ) \cdot {r}_{sd} \cdot {DPF}_{850 n m}\end{matrix} \qquad ( 4 )$$

thus leading to:

$$Δ {C}_{HbO 2} ( t ) = \frac{\begin{matrix}{ɛ}_{HHb , 730 n m} \cdot Δ {OD}_{850 n m} ( t ) \cdot \frac{1}{{r}_{sd} \cdot {DPF}_{850 nm}} - \\ - {ɛ}_{HHb , 850 n m} \cdot Δ {OD}_{730 n m} ( t ) \cdot \frac{1}{{r}_{sd} \times {DPF}_{730 n m}}\end{matrix}}{{ɛ}_{HHb , 730 n m} \cdot {ɛ}_{HbO 2 , 850} - {ɛ}_{HHb , 850 n m} \cdot {ɛ}_{HbO 2 , 730 n m}}$$

$$Δ {C}_{Hbb} ( t ) = \frac{\begin{matrix}{ɛ}_{HbO 2 , 730 n m} \cdot Δ {OD}_{850 n m} ( t ) \cdot \frac{1}{{r}_{sd} \cdot {DPF}_{850 nm}} - \\ {ɛ}_{HbO 2 , 850 n m} \cdot Δ {OD}_{730 n m} ( t ) \cdot \frac{1}{{r}_{sd} \times {DPF}_{730 n m}}\end{matrix}}{{ɛ}_{HbO 2 , 730 n m} \cdot {ɛ}_{HHb 2 , 850} - {ɛ}_{HbO 2 , 850 n m} \cdot {ɛ}_{HHb , 730 n m}} .$$

Accordingly, changes in the concentrations of oxy-Hb and deoxy-Hb can be computed based on reflected near-infrared light.

As described above with respect to FIGS. 6 and 8, multiple light detectors and/or multiple light sources can be used. According to one embodiment, ΔC_{HbO2}(t) and ΔC_{HHb}(t) are measured for each light detector and/or each optode.

In an example embodiment, the computed ΔC_{HbO2}(t) and ΔC_{HHb}(t), or changes in the concentrations of oxy-Hb and deoxy-Hb, are displayed on a display such as the display unit 170 in FIG. 1. The display unit 170 is configured to render information indicative of the efficacy of the stimulation. According to one embodiment, the display unit 170 is also capable to displaying parameters and/or configurations, such the intensity, duration, frequency, and/or phase of the stimulation administered.

In an embodiment, the system 100 may include a memory capable of storing information utilized in conjunction with performing stimulation. For example, the memory is capable of storing parameters and/or configurations associated with concurrently stimulating and detecting, including but not limited to, storing baseline blood oxygenation level values, baseline rCBF levels of one or more subjects, storing store programming for execution on the processing portion, or a combination thereof. In an embodiment, the information utilized in conjunction with performing stimulation may be stored in a separate computing device. The system 100 may send and retrieve such information via wired or wireless connections.

According to one embodiment, a tDCS threshold current intensity is quantified for a particular subject such as the subject 205. As described above, the effects of tDCS or other neuromodulation techniques on rCBF can be used to calculate the “normal” hemodynamic response to manipulation of the cortical excitability. For example, the subject 205 can be stimulated via tDCS at different current intensities, and for each current intensity, the corresponding influence on rCBF is measured and recorded. By correlating the current intensities and the corresponding effects, a dose-response relation for a particular subject, can be established. Based on the dose-response relation, an individualized tDCS threshold current intensity and/or an optimal tDCS configuration can be heuristically established.

According to other embodiments, one or more parameters of stimulation is adjusted based on the determined influence. For example, the effect of stimulation is determined based on a percentage and/or rate of change of the deoxy-Hb concentration. Based on the effect, parameters of stimulation may be maintained or altered to optimize stimulation. Parameters of stimulation may include, but not limited to, stimulation intensity such as current intensity of tDCS, polarity of stimulation such as anodal or cathodal, duration, a time interval of a pulse stimulation, oscillation frequency, oscillation phase, or the like.

According to another embodiment, a target stimulation influence on a subject can be received, for example, via an input device. For example, the system 100 receives a target concentration of deoxy-Hb, a target concentration oxy-Hb, a target concentration change of deoxy-Hb, and/or a target concentration change of oxy-Hb or the like. The system 100 can compute an individualized stimulation configuration for stimulating a subject such as the subject 205 that produces the target stimulation influence.

According to another embodiment, a “normal” hemodynamic response to manipulation of the cortical excitability is calculated. For example, the process 900 can be performed on a plurality of subjects. The effects of stimulation on the subjects can be analyzed to determine a typical or standard response to stimulation. This would be useful, for example, in chronic cerebrovascular disorders, to identify patients with a reduced capability to increase the blood flow on demand. In addition, this would also be useful, for example, in neurological and psychiatric disorders, to identify patients with a reduced or increased capability to regulate the blood flow on demand.

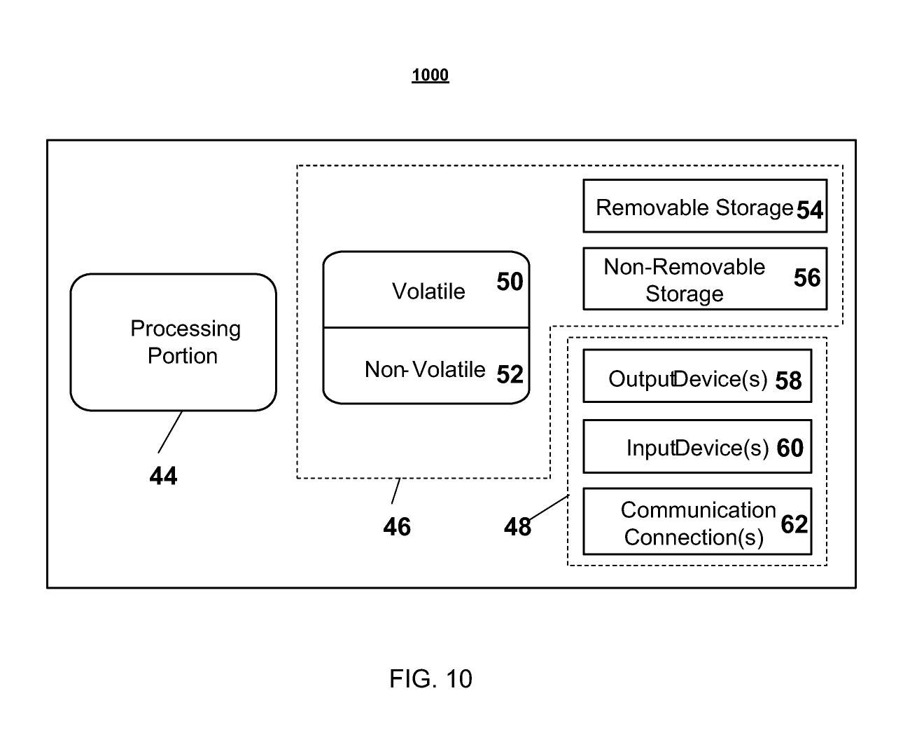

FIG. 10 is a block diagram of an example computing device 42 for concurrently stimulating and detecting. In an example configuration, the computing device 42 comprises various appropriate components of the system 100. It is emphasized that the block diagram depicted in FIG. 10 is exemplary and not intended to imply a specific implementation. Thus, the computing device 42 can be implemented in a single processor or multiple processors. Multiple processors can be distributed or centrally located. Multiple processors can communicate wirelessly, via hard wire, or a combination thereof.

The computing device 42 can be implemented as a client processor and/or a server processor. In a basic configuration, the computing device 42 comprises at least one processing portion 44, a memory portion 46, and an input/output portion 48. The processing portion 44, memory portion 46, and input/output portion 48 are operatively connected. The input/output portion 48 is capable of providing and/or receiving data associated with concurrently stimulating and detecting such as receiving a target stimulation influence for a subject, displaying parameters and/or configurations of stimulating and/or detecting, etc.

The processing portion 44 is capable of the operations associated with performing brain stimulation. For example, the processing portion 44 is capable of receiving a baseline regional blood oxygenation level, receiving a stimulated regional blood oxygenation level indicated by a reflected near-infrared light, wherein said stimulated regional blood oxygenation level is detected concurrently while stimulating a subject, comparing the stimulated regional blood oxygenation level with the baseline regional blood oxygenation level; and determining an influence of the stimulating on the subject based on the comparison, or a combination thereof.

The memory portion 46 is capable of storing any information utilized in conjunction with performing stimulation. For example, as described above, the memory portion 46 is capable of storing all parameters and/or configurations associated with concurrently stimulating and detecting, including but not limited to, storing baseline blood oxygenation level values, baseline rCBF levels of one or more subjects, storing store programming for execution on the processing portion, or a combination thereof. Depending upon the exact configuration and type of processor, the memory portion 46 can be volatile (such as RAM) 50, non-volatile (such as ROM, flash memory, etc.) 52, or a combination thereof. The computing device 42 can have additional features/functionality. For example, the computing device 42 can include additional storage (removable storage 54 and/or non-removable storage 56) including, but not limited to, magnetic or optical disks, tape, flash, smart cards or a combination thereof. The memory portion 46 can include volatile and nonvolatile, removable and non-removable media implemented in any method or technology for storage of information such as computer readable instructions, data structures, program modules, or other data. The memory portion 46 may include, but are not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, universal serial bus (USB) compatible memory, smart cards, or any other medium which can be used to store the desired information and which can be accessed by the processing portion 44. Any such computer storage media can be part of the processing portion 44.

The computing device 42 also can contain communications connection(s) 62 that allow the computing device 42 to communicate with other devices, for example. Communications connection(s) 62 can be connected to communication media. Communication media may embody computer readable instructions, data structures, program modules, or other data in a modulated data signal such as a carrier wave or other transport mechanism, and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. The term computer readable media as used herein includes both storage media and communication media. The computing device 42 also can have input device(s) 60 such as keyboard, mouse, pen, voice input device, touch input device, etc. Output device(s) 58 such as a display, speakers, printer, etc. also can be included.

While example embodiments of concurrently stimulating and detecting have been described in connection with various computing devices/processor, the underlying concepts can be applied to any computing device, processor, or system capable of performing stimulation. The various techniques described herein can be implemented in connection with hardware or software or, where appropriate, with a combination of both. Thus, the methods and apparatuses for performing stimulation, or certain aspects or portions thereof, can take the form of program code (i.e., instructions) embodied in tangible media, such as floppy diskettes, CD-ROMs, hard drives, or any other machine-readable storage medium, wherein, when the program code is loaded into and executed by a machine, such as a computer, the machine becomes an apparatus for performing stimulation. In the case of program code execution on programmable computers, the computing device will generally include a processor, a storage medium readable by the processor (including volatile and non-volatile memory and/or storage elements), at least one input device, and at least one output device. The program(s) can be implemented in assembly or machine language, if desired. The language can be a compiled or interpreted language, and combined with hardware implementations.

The methods and apparatuses for concurrently stimulating and detecting also can be practiced via communications embodied in the form of program code that is transmitted over some transmission medium, such as over electrical wiring or cabling, through fiber optics, or via any other form of transmission, wherein, when the program code is received and loaded into and executed by a machine, such as an EPROM, a gate array, a programmable logic device (PLD), a client computer, or the like, the machine becomes an apparatus for performing brain stimulation. When implemented on a general-purpose processor, the program code combines with the processor to provide a unique apparatus that operates to invoke the functionality of concurrently stimulating and detecting. Additionally, any storage techniques used in connection with concurrently stimulating and detecting can invariably be a combination of hardware and software.

While concurrently stimulating and detecting has been described in connection with the various embodiments of the various figures, it is to be understood that other similar embodiments can be used or modifications and additions can be made to the described embodiment for performing the same function of concurrently stimulating and detecting without deviating therefrom. Therefore, concurrently stimulating and detecting should not be limited to any single embodiment, but rather should be construed in breadth and scope in accordance with the appended claims.

1. A method comprising:
stimulating a central nervous system of a subject;
concurrently while stimulating, detecting near-infrared light reflected from the subject; and
determining an influence of the stimulating on the subject based on the detected reflected near-infrared light.

2. The method of claim 1, wherein the detected near-infrared light is indicative of a blood oxygenation level of the subject.

3. The method of claim 2, the determining further comprising:
comparing the blood oxygenation level indicated by the detected near-infrared light with a baseline blood oxygenation level; and
determining the influence of the stimulating based on a result of the comparing.

4-7. (canceled)

8. The method of claim 1, wherein said stimulating comprises direct current stimulation.

9. (canceled)

10. The method of claim 1, wherein said stimulating comprises magnetic stimulation.

11. The method of claim 1, the method further comprising:
heuristically determining a stimulating configuration based on the determined influence; and
stimulating the subject in accordance with the determined stimulating configuration.

12. The method of claim 1, the method further comprising:
receiving a target stimulation influence for the subject; and
computing an individualized stimulation configuration for the subject that corresponds to the target stimulation influence, based on the determined influence.

13-15. (canceled)

16. A device comprising:
a first portion that produces direct current stimulation;
a second portion that provides near-infrared light; and
a third portion that receives reflected near-infrared light, wherein the second portion and the third portion are connected to the first portion.

17. The device of claim 16, wherein the first portion, the second portion, and the third portion are configured to function concurrently.

18-21. (canceled)

22. The device of claim 16, further comprising a viscous conductive gel layer, the viscous conductive gel layer covering the first portion and leaving the second and third portion exposed.

23-24. (canceled)

25. The device of claim 16, wherein the second portion comprises a light source guide with a first end of the light source guide affixed to the first portion and a second end of the light source guide connected to a remote light source.

26. The device of claim 16, wherein the third portion comprises a light detection guide with a first end of the light detection guide affixed to the first portion.

27. The device of claim 16, wherein the first portion is flexible for adapting to contours of the subject for maintaining the second portion in an orientation orthogonal to a surface of subject.

28. The device of claim 16, further comprising a second detecting portion that is configured to detect an effect of an artifact of the near-infrared light.

29-31. (canceled)

32. A system for concurrently stimulating and detecting, the system comprising:
an apparatus configured to:
stimulate a central nervous system of a subject;
concurrently while stimulating, detect near-infrared light reflected from the subject; and
a processor configured to:
determine an influence of the stimulating on the subject based on the detected reflected near-infrared light.

33-45. (canceled)
